(19) United States
(12) Patent Application Publication
(10) Pub. No.: US 2006/0020342 A1
(43) Pub. Date: Jan. 26, 2006

(54) Facet-preserving artificial disc replacements

(76) Inventors: Bret A. Ferree, Cincinnati, OH (US); David Tompkins, Milford, OH (US); Alex B. Ferree, Cincinnati, OH (US)

Correspondence Address: John G. Posa;Gifford, Krass, Groh, Sprinkle,, Anderson & Citkowski, P.C., PO Box 7021, Troy, MI, 48007, US

(21) Appl. No.: 11/185,641

(22) Filed: Jul. 20, 2005

Related U.S. Application Data
(60) Provisional application No. 60/589,702, filed on Jul. 21, 2004.

(51) Int. Cl. A61F 2/44

(52) U.S. Cl. 623/17.15; 623/17.14; 606/96

(57) ABSTRACT

Artificial disc replacement (ADR) components cooperate to limit axial rotation and/or lateral bending to a greater degree when the spine is extended than when the spine is a neutral to flexed position, thereby decreasing loads placed upon the facet joints. In the preferred embodiment, truncated articulating surfaces with non-articulating side surfaces allow increasing amounts of axial rotation and lateral bending as the total disc replacement (TDR) moves from full extension to full flexion. Limiting axial rotation and lateral bending of the TDR protects the facet joints and the Annulus Fibrosus, since the facet joints carry more load when the spine is extended than when the spine is flexed. Thus the invention serves to protect the facet joints while allowing normal or near-normal spinal motion.

### REFERENCE TO RELATED APPLICATION

This application claims priority from U.S. Provisional Patent Application Ser. No. 60/589,702, filed Jul. 21, 2004, the entire content of which is incorporated herein by reference.

### FIELD OF THE INVENTION

This invention relates generally to artificial intervertebral disc replacement (ADR) and, in particular, to ADRs with facet-preserving features.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

Many spinal conditions, including degenerative disc disease, can be treated by spinal fusion or through artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion.

However, motion through an ADR also allows motion through the facet joints. Motion across arthritic facet joints could lead to pain following ADR. Some surgeons believe patients with degenerative disease and arthritis of the facet joints are not candidates for ADR. Current ADR designs do not attempt to limit the pressure across the facet joints or facet joint motion. Indeed, prior-art ADRs generally do not restrict motion. For example, some ADR designs place bags of hydrogel into the disc space which do not limit motion in any direction. In fact, ADRs of this kind may not, by themselves, provide sufficient distraction across the disc space. ADR designs with metal plates and polyethylene spacers may restrict translation but they do not limit the other motions mentioned above. The articular surface of the poly spacer is generally convex in all directions. Some ADR designs limit motion translation by attaching the ADR halves at a hinge.

One of the most important features of an ADR is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

The kinematics of ADRs are governed by the range of motion (ROM), the location of the center of rotation (COR) and the presence (or absence) of a variable center of rotation (VCOR). Generally ROM is limited by the facet joints and the AF. A natural disc has a VCOR, that is, the COR varies as the spine bends forward (flexion) and backward (extension). Typically, the vertebra above a natural disc translates forward 1-2 mm as the spine is flexed.

The facet joints limit axial rotation and lateral bending. The facet joints are most efficient in limiting these motions when the spine is extended. The lumbar facet joints are forced together as the spine extends and the joints separate as the spine is flexed. Arthritis of the facet joints frequently accompanies Degenerative Disc Disease (DDD). Arthritic facet joints may cause low back pain. Artificial Disc Replacement (ADR) primarily treats DDD. In fact, the increased spinal motion following ADR may increase the loads on the facet joints.

### SUMMARY OF THE INVENTION

This invention resides in artificial disc replacement (ADRs) offering various advantages, including a reduction of loading on the facet joints. The various components cooperate to limit axial rotation and to limit lateral bending to a greater degree when the spine is extended than when the spine is a neutral to flexed position. Limiting axial rotation and lateral bending of the TDR protects the facet joints and the Annulus Fibrosus since the facet joints carry more load when the spine is extended than when the spine is flexed. Thus, the invention decreases the loads placed upon the facet joints to protect the facet joints, while allowing normal or near-normal spinal motion.

In the preferred embodiment, the components include truncated convex and concave articulating surfaces with non-articulating side surfaces physically configured to maximize axial rotation and lateral bending at full flexion, to inhibit axial rotation in full extension, and to allow increasing amounts of axial rotation as the total disc replacement (TDR) moves from full extension to full flexion. The TDR may be configured to allow a certain degree of total lateral bending in the fully flexed position, between 6 and 20 degrees, for example, and 2.5 degrees or more axial rotation to the right and 2.5 degrees or more of axial rotation to the left when the TDR is fully flexed

### BRIEF DESCRIPTION OF THE DRAWINGS

FIG. 1A is a lateral view of a two-component ADR according to the present invention;

FIG. 1B is an anterior view of the ADR drawn in FIG. 1A;

FIG. 1C is an anterior view of the ADR drawn in FIG. 1B, showing how the ADR facilitates lateral bending of the spine;

FIG. 1D is a view of the top of the inferior ADR endplate (EP) drawn in FIG. 1A;

FIG. 1E is an exploded anterior view of a three-component ADR;

FIG. 1F is a view of the inferior surface of the spacer component drawn in FIG. 1E;

FIG. 2A is a lateral view of an alternative embodiment of an ADR;

FIG. 2B is a lateral view of the ADR drawn in FIG. 2A with the ADR drawn in a more extended position;

FIG. 2C is a lateral view of the ADR drawn in FIG. 2A with ADR in a more flexed position;

FIG. 2D is a view of the top of the ADR EP drawn in FIG. 2C;

FIG. 2E is a top view of an alternative embodiment wherein the articulating components are more posterior to those of FIG. 2D;

FIG. 2F is a top view of a different alternative embodiment wherein the articulating components are more closely spaced;

FIG. 2G is a top view of a further alternative embodiment including four articulating component;

FIG. 2H is a top view of yet a different alternative embodiment;

FIG. 3A is a top view of an ADR EP embodiment showing three concavities;

FIG. 3B is an anterior view of the ADR drawn in FIG. 3A;

FIG. 3C is a posterior view of the ADR drawn in FIG. 3B;

FIG. 4A is a lateral view of an alternative embodiment of the present invention;

FIG. 4B is a lateral view with the ADR drawn in FIG. 4A in an extended position;

FIG. 4C is an axial cross section of the convex projection from the inferior surface of the ADR drawn in FIG. 4A;

FIG. 4D is a view of the top of the concave articulating surface on the inferior ADR EP drawn in FIG. 4A;

FIG. 4E is an axial cross section of the convex and concave articulating components of the ADR drawn in FIG. 4A;

FIG. 4F is an axial cross section of the articulating components of the ADR drawn in FIG. 4E;

FIG. 5A is a view of the inferior surface of a further alternative embodiment of the present invention;

FIG. 5B is a view of the superior surface of an alternative embodiment of a concave articulating component;

FIG. 5C is an axial cross section through the articulating components drawn in FIGS. 5A and 5B;

FIG. 5D is an axial cross section through the articulating components drawn in FIG. 5C;

FIG. 6A is an axial cross section of an alternative shape of a convex articulating component which is smaller than the component drawn in FIG. 4C;

FIG. 6B is the view of the top of the concavity of the inferior ADR EP;

FIG. 6C is a lateral view of the superior ADR EP incorporating the articulating surface drawn in FIG. 6A;

FIG. 7A is an oblique view of an alternative embodiment of the invention drawn in FIG. 1A;

FIG. 7B is a lateral view of the embodiment of the invention drawn in FIG. 7A;

FIG. 7C is an anterior view of the embodiment of the invention drawn in FIG. 7A;

FIG. 7D is a view of the caudal portion of the embodiment of the invention drawn in FIG. 7A;

FIG. 7E is a view of the cranial portion of the embodiment of the invention drawn in FIG. 7A;

FIG. 7F is an oblique view of the cranial portion of the caudal TDR EP drawn in FIG. 7A;

FIG. 7G is an oblique view of the caudal portion of the cranial TDR EP drawn in FIG. 7A;

FIG. 8A is an axial cross section through the embodiment of the invention drawn in FIG. 7A;

FIG. 8B is an axial cross section of the embodiment of the invention drawn in FIG. 7A;

FIG. 8C is an axial cross section of the embodiment of the invention drawn in FIG. 7A;

FIG. 8D is an axial cross section of the embodiment of the invention drawn in FIG. 7A;

FIG. 8E is an axial cross section of the embodiment of the invention drawn in FIG. 7A;

FIG. 9 is an oblique view of an alternative embodiment of the invention;

FIG. 10A is an oblique view of the embodiment of the invention drawn in FIG. 7A;

FIG. 10B is an oblique view of an alternative embodiment of the invention;

FIG. 10C is an oblique view of the bottom of the embodiment of the TDR drawn in FIG. 10A and the tips of the instrument drawn in FIG. 10B;

FIG. 10D is an oblique view of the top of the TDR drawn in FIG. 10A and the tips of the instrument drawn in FIG. 10B;

FIG. 10E is an oblique view of the anterior surface of the embodiment of the invention drawn in FIG. 10D;

FIG. 11A is an oblique view of an alternative embodiment of the invention;

FIG. 11B is an oblique view of the anterior portion of the TDR drawn in FIG. 7A and the tip of the drill guide drawn in FIG. 11A;

FIG. 11C is an oblique view of the anterior portions of the embodiment of the invention drawn in FIG. 11B;

FIG. 11D is a lateral view of the embodiment of the invention drawn in FIG. 11C;

FIG. 11E is a sagittal cross section of the embodiment of the invention drawn in FIG. 11D;

FIG. 12A is an oblique view of an alternative embodiment of the invention drawn in FIG. 7A;

FIG. 12B is an oblique view of the tip of a fixation component drawn in FIG. 12A;

FIG. 12C is an oblique view of the tip of an alternative embodiment of the invention drawn in FIG. 12B; and

FIG. 12D is an enlarged, oblique view of the tip of the fixation component drawn in FIG. 12C and a portion of the TDR drawn in FIG. 7A.

### DETAILED DESCRIPTION OF THE INVENTION

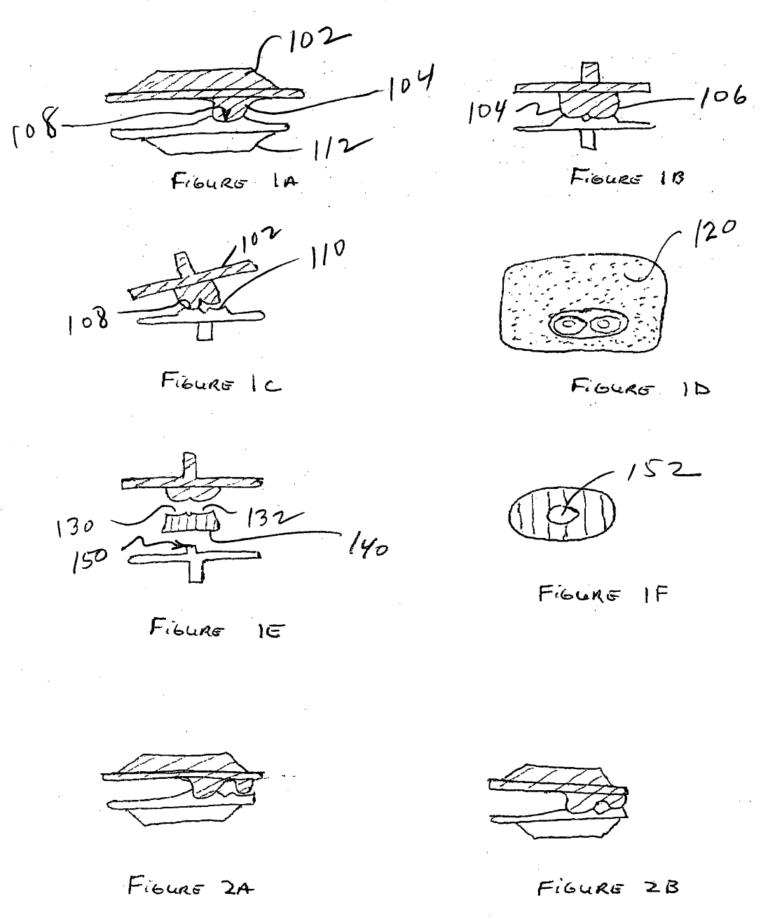

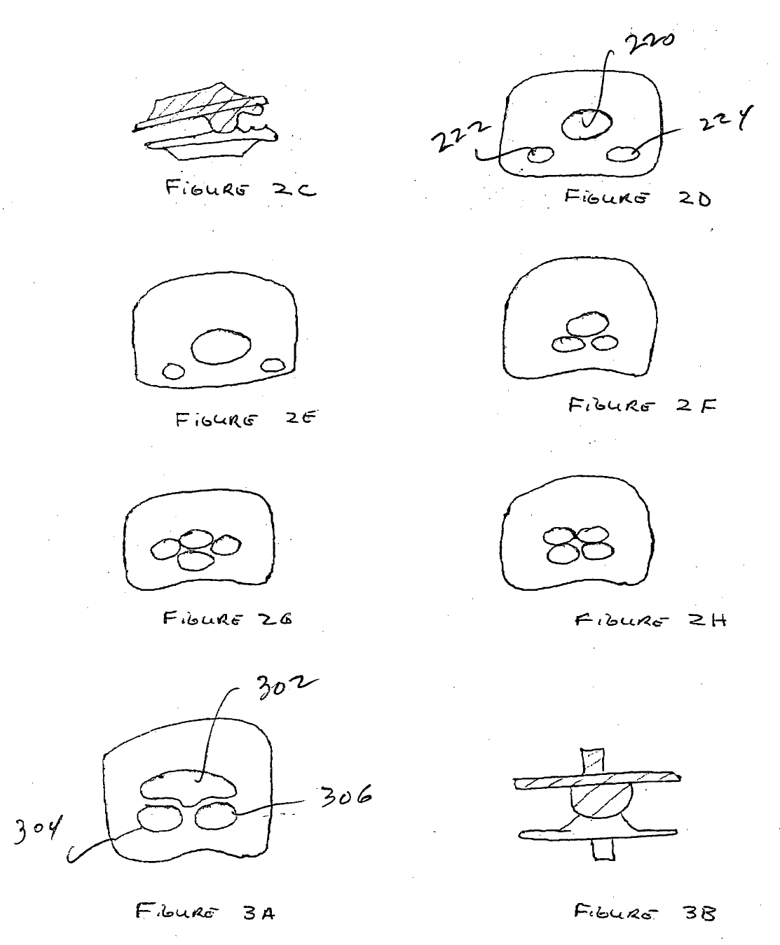

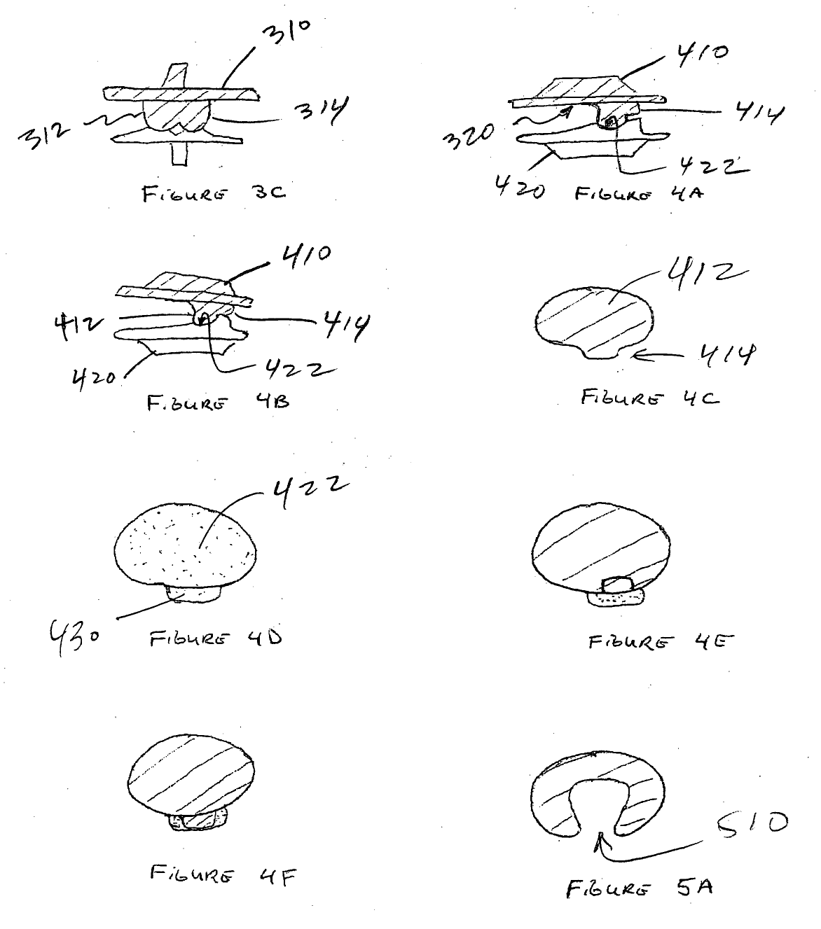

FIG. 1A is a lateral view of a two-component ADR according to the invention. The superior component 102 includes a set of laterally spaced projections 104, 106 that co-act with respective concavities 108, 110 in an inferior component 112. FIG. 1B is an anterior view of the ADR drawn in FIG. 1A. FIG. 1C is an anterior view of the ADR drawn in FIG. 1B, showing how the ADR facilitates lateral bending of the spine. The ADR articulates in the adjacent convexity and concavity when the ADR allows lateral bending to the opposite side. The ADR articulates through both sets of concavities and concavities during flexion and extension while resisting axial rotation.

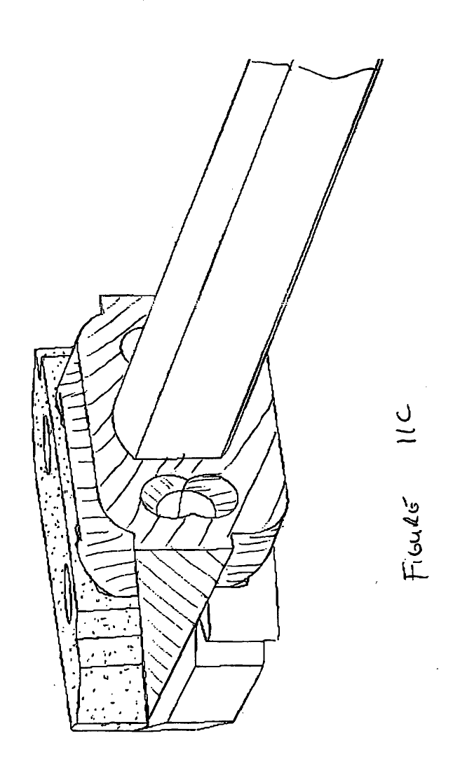

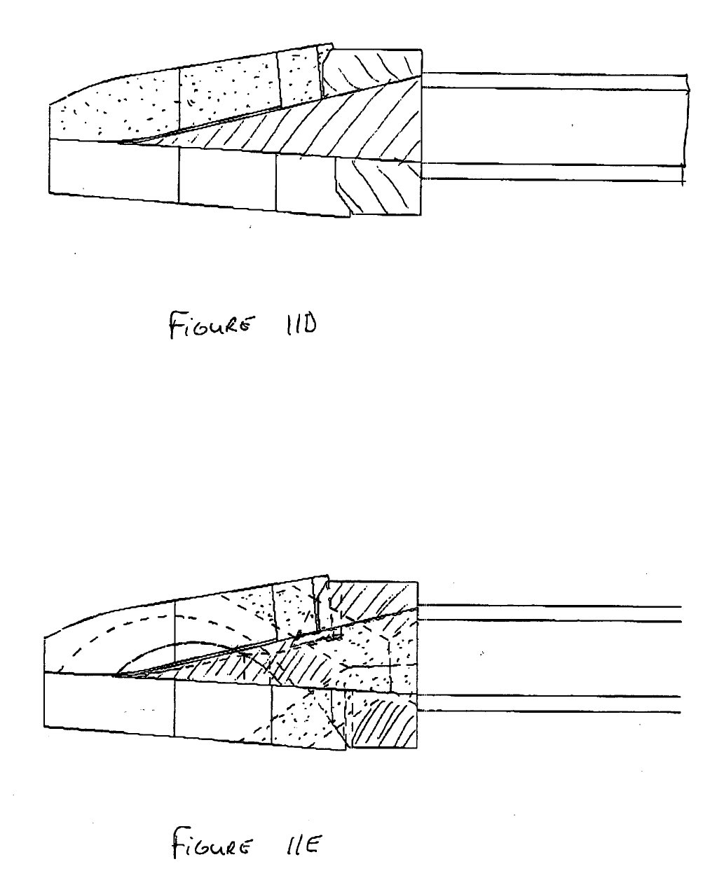

FIG. 1D is a view of the top of the inferior ADR endplate (EP) drawn in FIG. 1A. Area 120 represents a cushion material. This embodiment of the ADR is similar to the ADR taught in my co-pending application U.S. Ser. No. 10/434,930 (see FIG. 10B). The restricted motion of the ADR helps protect the cushion material. The cushion material could be any appropriate compressible and/or resilient material, including elastomers, hydrogels, or polymers.

FIG. 1E is an exploded anterior view of a three-component ADR, wherein the concavities 130, 132 lie on a spacer component 140. As taught in my co-pending application U.S Provisional Patent Application Ser. No. 60/518,971, the spacer component may permit at least some axial rotation. The spacer component may also permit as least some translation. FIG. 1F is a view of the inferior surface of the spacer component drawn in FIG. 1E. The post 150 of the inferior ADR EP fits into the hole 152 in the inferior surface of the spacer component 140.

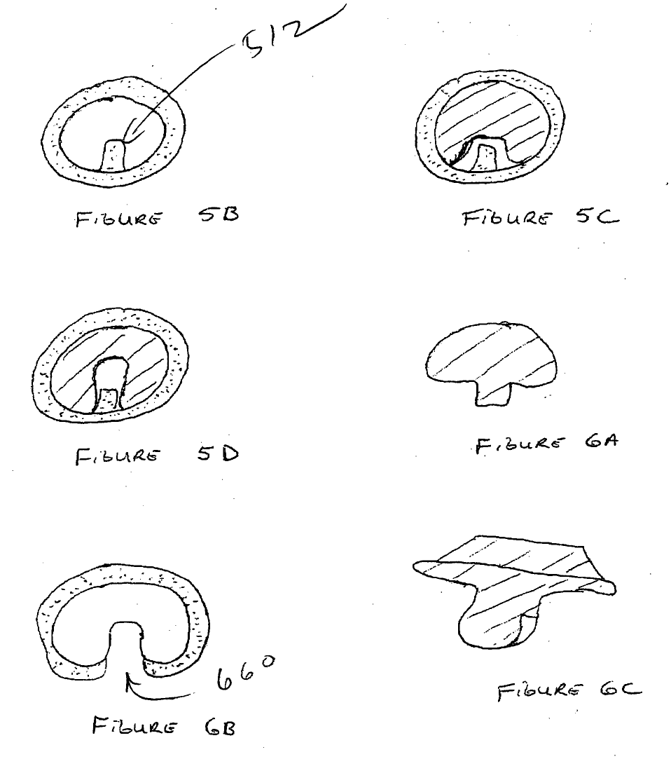

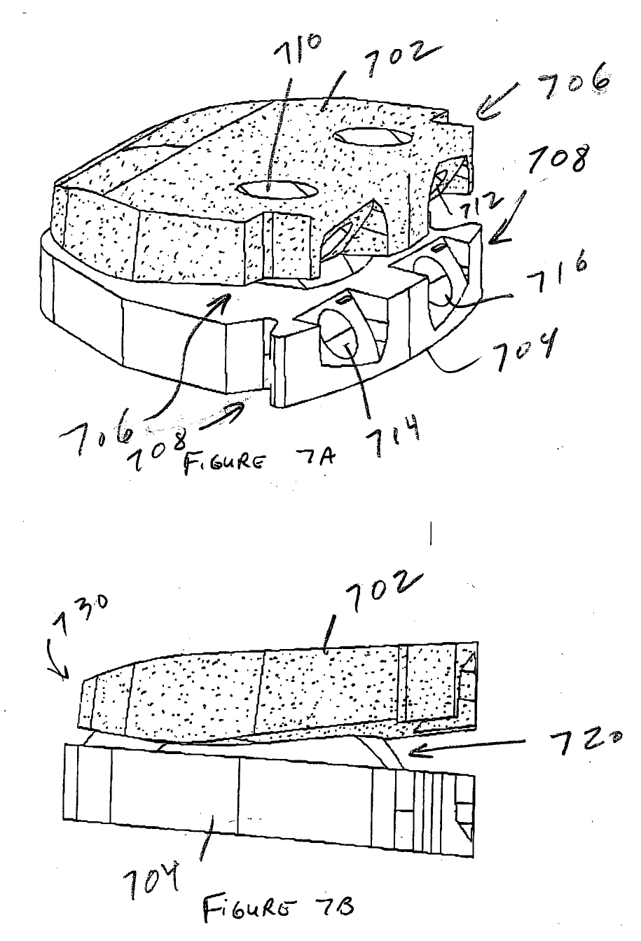

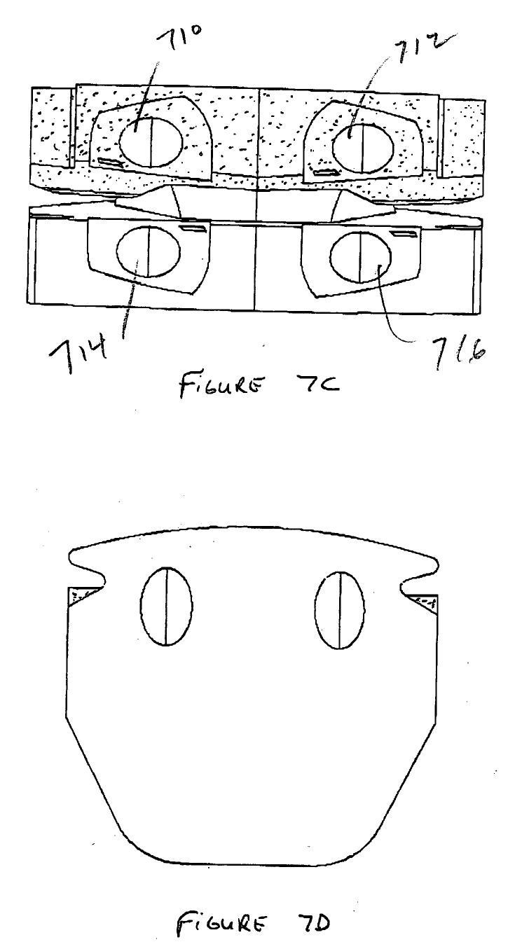

FIG. 2A is a lateral view of an alternative embodiment of an ADR that articulates through an anterior set or sets of concavities and convexities while the ADR is in a neutral to flexed position. FIG. 2B is a lateral view of the ADR drawn in FIG. 2A with the ADR drawn in a more extended position than was drawn in FIG. 2A. A posterior set or sets of concavities and convexities cooperate in extension to reduce or eliminate axial rotation and/or lateral bending. FIG. 2C is a lateral view of the ADR drawn in FIG. 2A with ADR in a more flexed position than was drawn in FIG. 2A. The space between the posterior set or sets of concavities and convexities permit increased lateral bending and increased axial rotation of the ADR in the flexed position.

FIG. 2D is a view of the top of the ADR EP drawn in FIG. 2C. The larger, anterior, circle 220 may represent a spherical concavity. The smaller, posterior, circles 222, 224 may represent spherical concavities or cone-shaped depressions. FIG. 2E is a top view of an alternative embodiment wherein the articulating components are more posterior to those of FIG. 2D. FIG. 2F is a top view of a different alternative embodiment wherein the articulating components are more closely spaced. FIG. 2G is a top view of a further alternative embodiment including four articulating components. FIG. 2H is a top view of yet a different alternative embodiment.

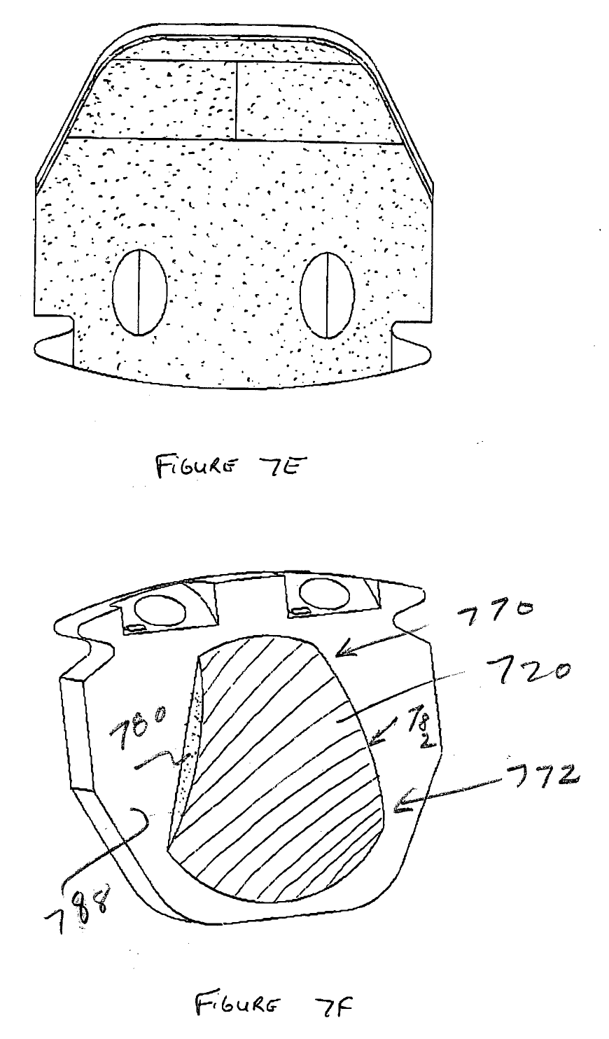

FIG. 3A is a top view of an ADR EP embodiment which has three concavities 302, 304, 306. FIG. 3B is an anterior view of the ADR drawn in FIG. 3A. FIG. 3C is a posterior view of the ADR drawn in FIG. 3B. The posterior portion of the articulating component of the upper ADR EP 310 includes at least two spherical projections 312, 314. The two spherical projections transition into a single convex surface 320 as they reach the anterior portion of the component.

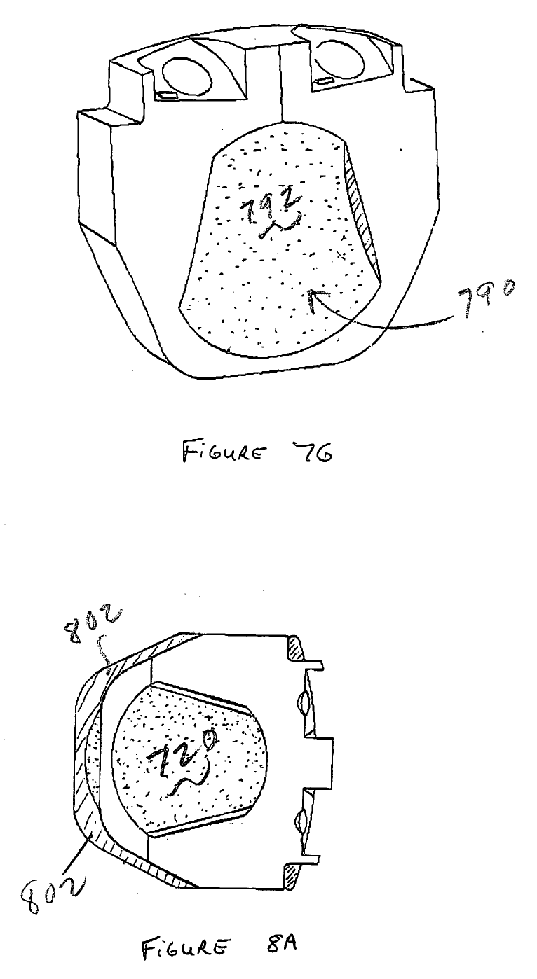

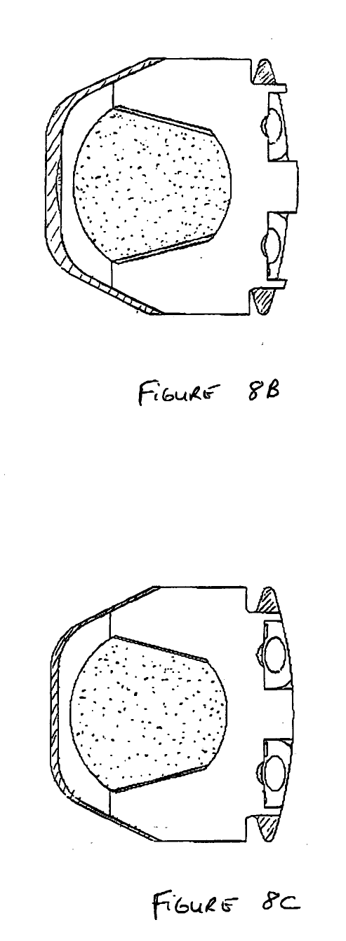

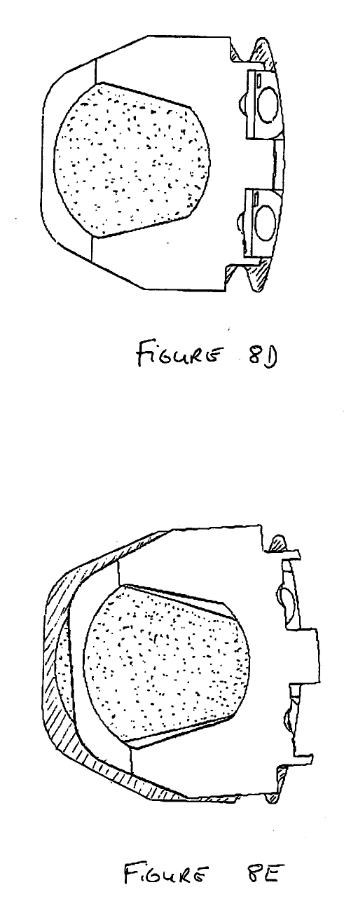

FIG. 4A is a lateral view of an alternative embodiment wherein the superior ADR EP 410 has a convex articulating component 412 and a projection 414 from its posterior surface. The inferior ADR EP 420 has a corresponding concave articulating surface 422. The projection 414 from the superior convex articulating component lies above the inferior ADR EP when the ADR is in a neutral to flexed position. FIG. 4B is a lateral view with the ADR drawn in FIG. 4D in an extended position. The projection from the convex component fits into a notch 430 in the concave component. The two components cooperate to limit lateral bending and to limit axial rotation when the ADR is extended.

FIG. 4C is an axial cross section of the convex projection from the inferior surface of the ADR drawn in FIG. 4A. FIG. 4D is a view of the top of the concave articulating surface on the inferior ADR EP drawn in FIG. 4A. FIG. 4E is an axial cross section of the convex and concave articulating components of the ADR drawn in FIG. 4A taken with the ADR in a neutral to flexed position. FIG. 4F is an axial cross section of the articulating components of the ADR drawn in FIG. 4E. The cross section was taken with the ADR in an extended position. The projection from the superior articulating surface lies within the notch in the concave articulating surface, thus limiting axial rotation and lateral bending of the ADR in extension.

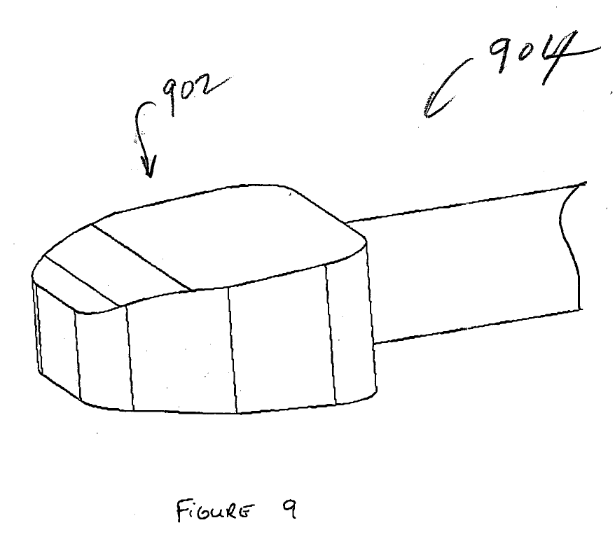

FIG. 5A is a view of the inferior surface of an alternative embodiment of a convex articulating component having a notch or a concavity 510. FIG. 5B is a view of the superior surface of an alternative embodiment of a concave articulating component which has a projection 512. FIG. 5C is an axial cross section through the articulating components drawn in FIGS. 5A and 5B. The cross section was taken through the ADR with the ADR in a neutral to flexed position. Space between the projection and the concavity allows axial rotation and lateral bending of the ADR when the ADR is in a neutral to flexed position. FIG. 5D is an axial cross section through the articulating components drawn in FIG. 5C taken through the ADR with the ADR in an extended position. The decreased space between the projection and the concavity in the extended ADR decreases the allowed axial rotation and lateral bending of the ADR.

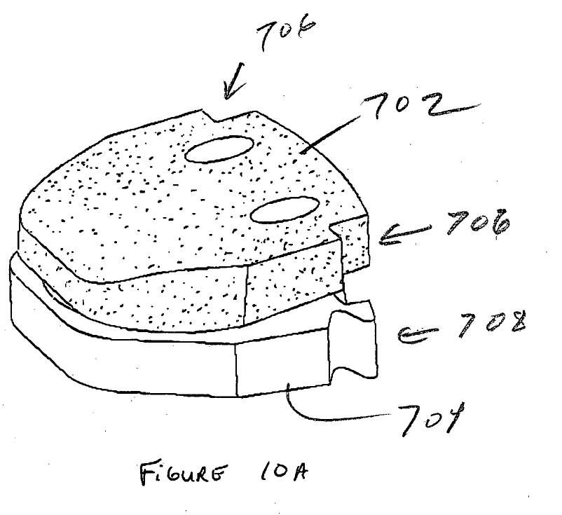

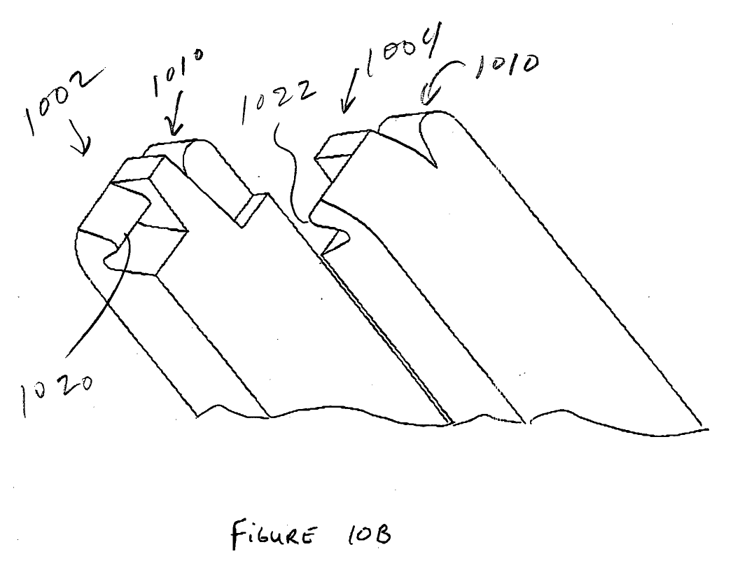

FIG. 6A is an axial cross section of an alternative shape of a convex articulating component which is smaller than the component drawn in FIG. 4C. The component is less than a sphere. FIG. 6B is the view of the top of the concavity of the inferior ADR EP that cooperates with the articulating component drawn in FIG. 6A. The concavity has notch 660 that cooperates with the projection from the articulating component drawn in FIG. 6A. FIG. 6C is a lateral view of the superior ADR EP that incorporates the articulating surface drawn in FIG. 6A.

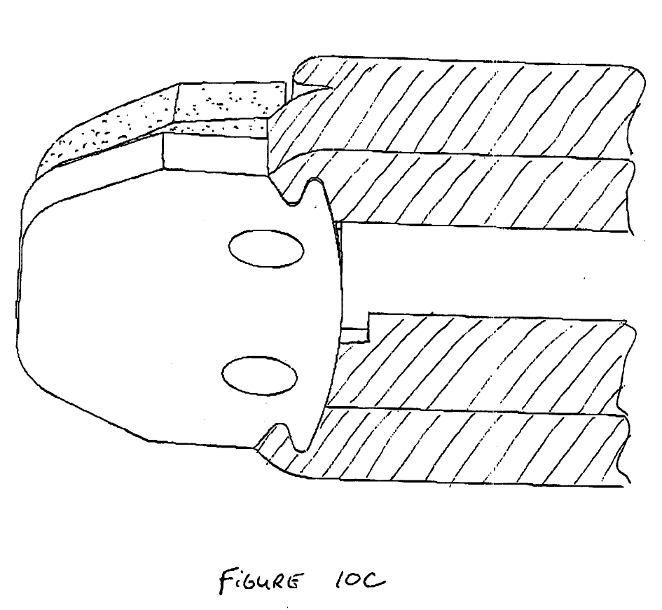

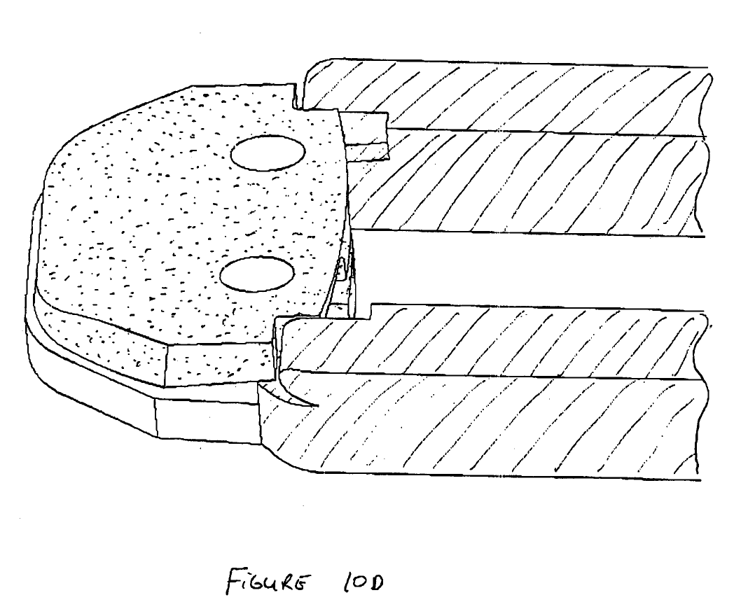

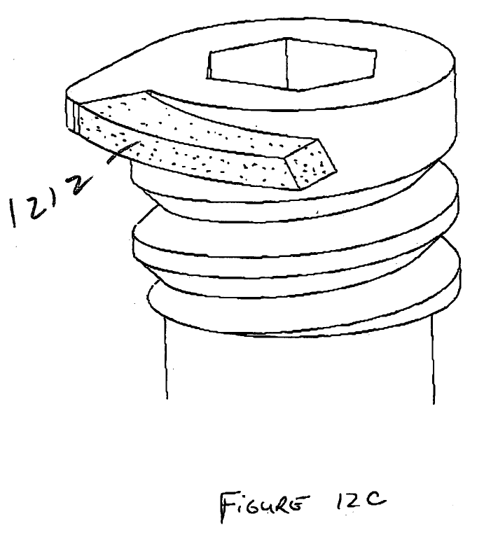

FIG. 7A is an oblique view of an alternative embodiment of the invention. The two-component total disc replacement (TDR) has features 706, 708 on the anterior portion of the device to cooperate with insertion tools, as explained in further detail below. The anterior portions of the superior/cranial and inferior/caudal endplates 702, 704 also have holes 710, 712 and 714, 716 for insertion of fixation components 1202, 1204, 1206, 1208 (see FIG. 12A). FIG. 7B is a lateral view of the embodiment of the invention drawn in FIG. 7A. A truncated convexity 720 projects from the caudal TDR EP 704. The cranial TDR EP 702 has a corresponding truncated concavity on the side of the TDR EP that faces the caudal TDR EP. In the preferred embodiment, the convexity and concavity include spherical articulating surfaces.

The caudal surface of the cranial TDR EP has surfaces with more than one plane. The caudal surface of the cranial TDR EP is preferably has three planes. The drawing illustrates the cranial TDR EP 702 is tallest in the posterior portion of the TDR. The TDR EPs are configured to strike across the full posterior portions of the EPs when the TDR is in full extension. The configuration limits extension of the TDR and prohibits lateral bending of the TDR, when the TDR is in full extension. The assembled TDR is a modified trapezoid in shape.

A narrow portion 730 of the cranial TDR EP permits posterior placement of cervical embodiments of the invention without removal of the lip of bone that projects from the posterior portion of the caudal vertebral endplate (VEP) of the vertebra cranial to the TDR. Lumbar embodiments of the invention include a narrow dorsal portion of the caudal TDR EP to permit posterior placement of the TDR without removal of lip of bone that projects from posterior aspect of the cranial VEP of the vertebra caudal to the TDR.

Cervical embodiments of the invention are preferably between 4 mm and 14 mm in height. For example, the posterior aspect of the cervical embodiment of the invention may be 5 mm tall and the anterior portion of the device may be 7 mm tall. Lumbar embodiments of the invention are preferably between 5 mm and 22 mm in height. For example, the posterior aspect of the lumbar embodiment of the invention may be 12 mm tall and the anterior portion of the device may be 15 mm tall. Cervical embodiments of the invention are preferably 10 mm to 22 mm wide. Lumbar embodiments of the invention are preferably 30 to 60 mm wide. Cervical embodiments of the invention are preferably 10 to 22 mm long in the anterior to posterior direction. Lumbar embodiments of the invention are preferably 20 to 36 mm long in the anterior to posterior direction.

FIG. 7C is an anterior view of the embodiment of the invention drawn in FIG. 7A. Each TDR EP has two holes 710, 712 and 714, 716. The dorsal portion of the cranial TDR EP is narrower than the ventral portion of the cranial TDR EP. The TDR EPs are configured to strike along the sagittal midline when the TDR is in full flexion. The configuration limits flexion of the TDR and permits limited lateral bending, when the TDR is in full flexion.

FIG. 7D is a view of the caudal portion of the embodiment of the invention drawn in FIG. 7A. FIG. 7E is a view of the cranial portion of the embodiment of the invention drawn in FIG. 7A. FIG. 7F is an oblique view of the cranial portion of the caudal TDR EP drawn in FIG. 7A. The truncated spherical convexity projects from the cranial side of the caudal TDR EP. The spherical projection is narrower on the anterior portion 770 of the device than it is on the posterior portion 772 of the device. The sides 780, 782 of the truncated portions of the spherical convexity are preferably flat and perpendicular to the horizontal plane 788 of the TDR EP. The spherical articulating surfaces of the cervical and lumbar embodiments of the invention are preferably made with a radius between 8 mm and 36 mm. The most preferred cervical embodiment of the invention uses a radius of between 16 and 17 mm; 16.6 mm in particular. The most preferred lumbar embodiment of the invention uses a radius on the order of 18 mm.

FIG. 7G is an oblique view of the caudal portion of the cranial TDR EP drawn in FIG. 7A. The TDR EP has a truncated spherical concavity 790. The spherical surface 792 of the concavity is configured to be congruent with the spherical convex surface of the caudal TDR EP.

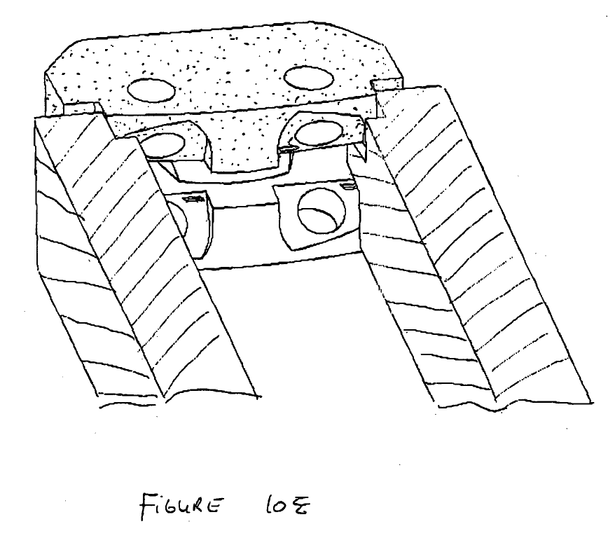

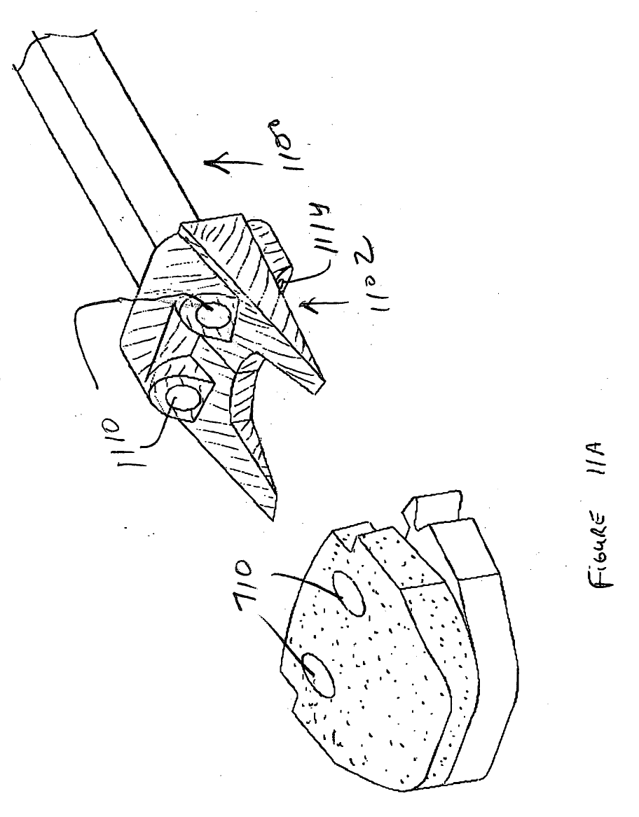

FIG. 8A is an axial cross section through the embodiment of the invention drawn in FIG. 7A. Area 720 represents the spherical convexity. The area 802 represents the non-spherical portion of the cranial side of the caudal TDR EP. The cross section was taken with the TDR in full flexion. For example, the TDR may be in 10 degrees of flexion. The truncated spherical articulating surfaces are configured to allow maximal axial rotation when the TDR is fully flexed. For example, the truncated spherical articulating surfaces may 2.5 degrees or more of axial rotation to the right and 2.5 degrees or more of axial rotation to the left when the TDR is fully flexed. However, the truncated sides of the spherical articulating surfaces impinge to limit axial rotation. The space between the truncated surfaces permits axial rotation when the TDR is fully flexed.

FIG. 8B is an axial cross section of the embodiment of the invention drawn in FIG. 7A, taken with the TDR in less-than-full flexion. The space between the truncated portions of the spherical articulating surfaces is less than the space between the truncated portions of the spherical articulating surfaces of the fully flexed TDR drawn in FIG. 8A. Consequently, the partially flexed TDR will not permit as much axial rotation as the fully flexed TDR. For example, the TDR may permit 2 degrees of axial rotation to the left and 2 degrees to the right in the position drawn in FIG. 8B.

FIG. 8C is an axial cross section of the embodiment of the invention drawn in FIG. 7A exhibiting more extension than the TDR drawn in FIG. 8B. The space between the truncated portions of the spherical articulating surfaces is even less than the space between the truncated portions of the spherical articulating surfaces of the partially flexed TDR drawn in FIG. 8B. The partially extended TDR allows less axial rotation than the partially flexed TDR drawn in FIG. 8B. For example, the partially extended TDR drawn in FIG. 8C may allow 1 degree of axial rotation to the left and 1 degree of axial rotation to the right.

FIG. 8D is an axial cross section of the embodiment of the invention drawn in FIG. 7A in full extension. The truncated surfaces of the spherical convexity now impinge against the truncated surfaces of the spherical concavity to inhibit axial rotation. FIG. 8E is an axial cross section of the embodiment of the invention drawn in FIG. 7A. The TDR was drawn in full flexion. The drawing illustrates impingement of the truncated surfaces to limit axial rotation.

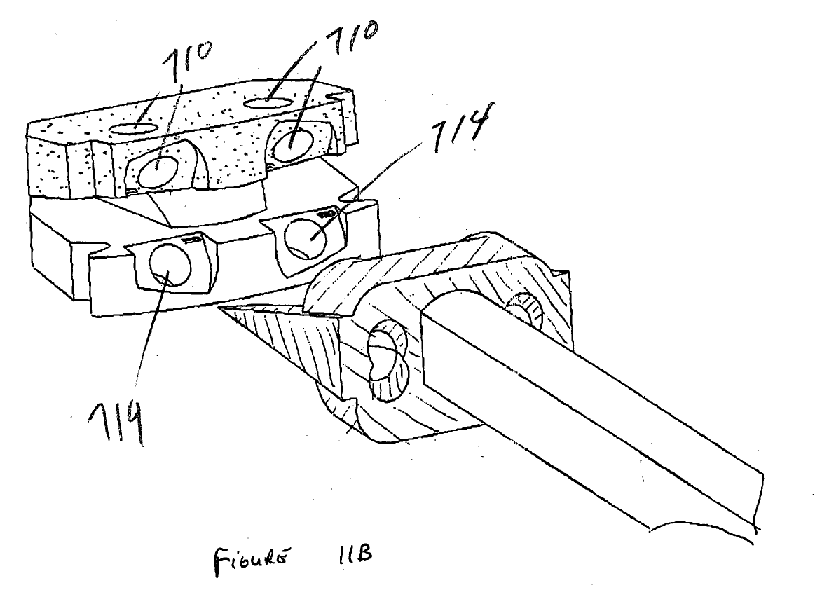

FIG. 9 is an oblique view of a “plug distractor” according the invention. The tip 902 of the instrument 904 may be impacted into the disc space to separate the vertebrae. The tip of the instrument is shaped similar to the assembled TDR drawn in FIG. 7A. Alternative embodiment of the invention may be used to machine the Vertebral Endplates (VEPs) to receive the TDR. For example, the surfaces of the tip of the “broach” may incorporate cutting flutes. The cutting flutes may be incorporated into the top, bottom, and sides of the tip of the instrument. The cutting flutes are limited to the cranial side of the instrument in the preferred embodiment of the invention. In general, the caudal VEP of the cranial vertebra requires more machining than the cranial VEP of the vertebra caudal to the TDR.

FIG. 10A is an oblique view of the embodiment of the invention drawn in FIG. 7A. As discussed in conjunction with FIG. 7A, the anterior portion of the TDR is configured to cooperate with an instrument to insert the TDR. The upper TDR EP 702 incorporates different features 706 than the lower TDR EP 704 (708).

FIG. 10B is an oblique view of a pliers-like insertion instrument according to the invention. The tips 1002, 1004 of the instrument are configured to attach to the anterior portion of the embodiment of the TDR drawn in FIG. 10A. The upper portions 1010, 1012 of the tips fit against the sides of the upper TDR EP. The lower portions of have triangular projections 1020, 1022 that fit into the triangular recesses of the lower TDR EP.

FIG. 10C is an oblique view of the bottom of the embodiment of the TDR drawn in FIG. 10A and the tips of the instrument drawn in FIG. 10B. The triangular projections from the instrument fit into triangular recesses in the lower TDR EP. The configuration allows the instrument to be used to insert and to remove the TDR from the disc space.

FIG. 10D is an oblique view of the top of the TDR drawn in FIG. 10A and the tips of the instrument drawn in FIG. 10B. FIG. 10E is an oblique view of the anterior surface of the embodiment of the invention drawn in FIG. 10D. The flat sides of the instrument fit against the flat sides of the upper TDR EP. The configuration allows the TDR to flex or extend somewhat during insertion of the TDR into the disc space. Small changes in TDR flexion or extension facilitate TDR insertion. The ends of the tips of the upper portion of the instrument may be configured to strike the anterior portion of the upper TDR EP along the surfaces of the upper TDR EP perpendicular and adjacent to the surfaces of the upper TDR EP grasped by the pliers-like instrument to limit the amount of TDR flexion or extension allowed by the instrument. For example, the instrument and the TDR may cooperate to allow the TDR to move from full extension to 5 degrees of flexion during insertion of the TDR into the disc space.

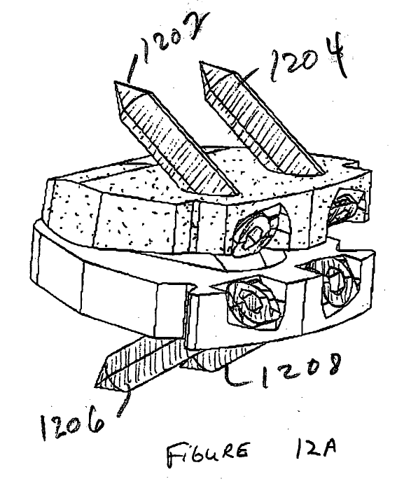

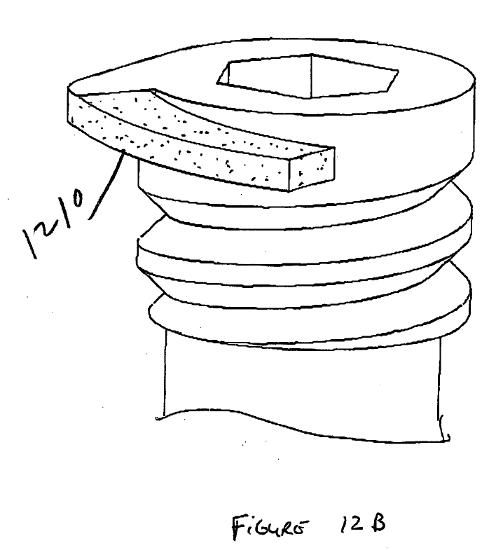

FIG. 11A is an oblique view of a drill guide instrument 1100 according to the invention. The tip of a drill guide was drawn to the right of the anterior surface of the TDR drawn in FIG. 7A. The drill guide has a wedge shaped component 1102 that fits between the TDR EPs. The wedge-shaped component straddles the spherical convexity from the lower TDR EP.

FIG. 11B is an oblique view of the anterior portion of the TDR drawn in FIG. 7A and the tip of the drill guide drawn in FIG. 11A. Oblique holes 1110, 1114 through the guide align with the holes that course obliquely through the TDR EPs. The drill guide has four holes on the TDR side of the device and two holes on the handle side of the instrument. The holes that pass obliquely through the drill guide connect on the handle side of the instrument. FIG. 11C is an oblique view of the anterior portions of the embodiment of the invention drawn in FIG. 11B. The drill guide has been forced into the space between the TDR EPs. FIG. 11D is a lateral view of the embodiment of the invention drawn in FIG. 11C.

FIG. 11E is a sagittal cross section of the embodiment of the invention drawn in FIG. 11D. The dotted areas represent the holes on one side of the TDR and the instrument. The holes in the instrument align with the holes in the TDR. The instrument may be used to guide drill bits and/or fixation components into the TDR. The drill guide may also be used to place the TDR in full extension to facilitate drill bit and/or fixation member placement.

FIG. 12A is an oblique view of an alternative embodiment of the invention drawn in FIG. 7A. Four fixation components 1202, 1204, 1206, 1208 have been placed into the TDR. The upper fixation members, spikes or screws, diverge from the lower fixation members. The upper set of fixation members may also diverge or converge with one another. Similarly, the lower set of fixation members may diverge or converge with one another.

FIG. 12B is an oblique view of the end of a preferred fixation component according to the invention including a projection 1210 that cooperates with a wedge-shaped projection on the TDR EP to prevent the fixation from backing out of the TDR. FIG. 12C is an oblique view of an alternative tip configuration according to the invention.

FIG. 12D is an enlarged, oblique view of the tip of the fixation component drawn in FIG. 12C and a portion of the TDR drawn in FIG. 7A. The projection 1212 from the fixation member passes over a wedge 1214 on the TDR EP. The elasticity of the projection pulls the projection against the TDR EP after it passes over the wedge on the TDR EP. The end of the projection impinges against the vertical wall of the wedge to inhibit rotation of the fixation member in the direction opposite to the direction of rotation used to insert the fixation component.

We claim:

1. An artificial disc replacement (ADR) system, comprising:
a superior component having a first articulating surface;
an inferior component having a second articulating surface;
both components having anterior and posterior portions; and
wherein the components are physically configured to limit axial rotation, lateral bending, or both, to a greater degree during extension as compared to flexion.

2. The system of claim 1, wherein the components are physically configured to inhibit axial rotation, lateral bending, or both, at full extension.

3. The system of claim 1, wherein the components are physically configured to maximize axial rotation, lateral bending, or both, at full flexion.

4. The system of claim 1, wherein the components are physically configured to provide a progressive change in the degree of axial rotation, lateral bending, or both, as articulation proceeds from full extension to full flexion.

5. The system of claim 1, wherein:
the first articulating surface includes a convex portion; and
the second articulating surface includes a concave portion.

6. The system of claim 1, wherein:
the first articulating surface includes a convex spherical portion; and
the second articulating surface includes a concave spherical portion.

7. The system of claim 1, wherein:
the first articulating surface forms part of a truncated convexity having non-articulating sidewalls that are spaced apart to a greater degree posteriorly as compared to anteriorly; and
the second articulating surface forms part of a truncated concavity having non-articulating sidewalls that are spaced apart to a greater degree posteriorly as compared to anteriorly.

8. The system of claim 1, wherein:
the first articulating surface forms part of a truncated convexity having first non-articulating sidewalls that are spaced apart to a greater degree posteriorly as compared to anteriorly;
the second articulating surface forms part of a truncated concavity having second non-articulating sidewalls that are spaced apart to a greater degree posteriorly as compared to anteriorly;
the first and second sidewalls are spaced apart by a gap during flexion; and
wherein the gap closes as articulation proceeds from flexion to extension.

9. The system of claim 1, wherein:
the first articulating surface forms part of a truncated spherical convexity having first non-articulating sidewalls that are spaced apart to a greater degree posteriorly as compared to anteriorly;
the second articulating surface forms part of a truncated spherical concavity having second non-articulating sidewalls that are spaced apart to a greater degree posteriorly as compared to anteriorly;
the first and second sidewalls are spaced apart by a gap during flexion; and
wherein the gap closes as articulation proceeds from flexion to extension.

10. The system of claim 1, wherein the components are physically configured to allow 6 to 20 degrees of lateral bending during flexion.

11. The system of claim 1, wherein the components are physically configured to allow 2.5 degrees or more of axial rotation to the right and 2.5 degrees or more of axial rotation to the left during flexion.

12. The system of claim 1, wherein the anterior portions of the components include geometric shapes to receive an insertion tool.

13. The system of claim 1, wherein:
the anterior portions of the components include geometric shapes;
an insertion tool including tip with geometric shapes corresponding to those of the anterior portions of the components.

14. The system of claim 1, wherein the anterior portions of the components include through-bores to receive screws or other fasteners.

15. The system of claim 1, wherein:
the components include through-bores to receive screws or other fasteners; and
an instrument with through-bores corresponding to those of the components thereby creating a drill guide when the instrument is in contact with the components.

16. The system of claim 1, wherein the articulating surfaces are spherical, having radii in the range of 8 mm to 36 mm.

17. The system of claim 1, wherein the components have a height in the range of 4 mm to 22 mm.

18. The system of claim 1, wherein the components have a width in the range of 10 mm to 60 mm.

19. The system of claim 1, wherein the components have an anterior-to-posterior in the range of 10 mm to 36 mm.

20. The system of claim 1, wherein the components have different dimensions to suit placement at different spinal levels.
